(19) United States
(12) Patent Application Publication
(10) Pub. No.: US 2024/0363256 A1
(43) Pub. Date: Oct. 31, 2024

(54) METHOD AND SYSTEM FOR SELECTING A CLINICAL PATHWAY

(71) Applicant: XWAVE TECHNOLOGIES LIMITED, Dublin 2 (IE)

(72) Inventors: Ronan KILLEEN, Dublin 4 (IE); Mitchell O'GORMAN, Dublin 6w (IE); Richard Simon CARBAJO, Dublin 15 (IE)

(21) Appl. No.: 18/681,707

(22) PCT Filed: Aug. 10, 2022
(86) PCT No.: PCT/IB2022/057469

§ 371 Date: Feb. 6, 2024

(30) Foreign Application Priority Data
Aug. 10, 2021 (EP) 21190693.8
Sep. 9, 2021 (GB) 2112892.1

(51) Int. Cl. G16H 50/70; G16H 10/40; G16H 15/00; G16H 50/20

(52) U.S. Cl. CPC G16H 50/70; G16H 10/40; G16H 15/00; G16H 50/20

(57) ABSTRACT

A clinical decision support system and method provides a practitioner with an indication of individual specific prospective usefulness of a referral. The practitioner is provided with individual-specific indication of prospective usefulness of findings for a referral, wherein comparative and statistical input data is supplemented by nuanced data to predict the usefulness of findings from optional and/or select subsequent diagnostic tests relative to individual specific propensity for a condition/disease.

### FIELD OF THE INVENTION

The present invention relates to the field of medicine and in particular to a system and method to provide an indication of individual person specific prospective usefulness of a referral.

### BACKGROUND

Diagnostic imaging refers to non-invasive methods for identifying and monitoring disease. Among diagnostic imaging techniques are x-ray, nuclear (use of radioactive materials), ultrasound, magnetic resonance imaging (MRI), computed tomography (CT), fluoroscopy and positron emission tomography (PET). Each have advantages and disadvantages.

The use of one or more imaging procedures throughout the course of a diagnostic procedure informs a practitioner's decisions: for example, to identify regions of interest indicative of a propensity for a disease, to identify early-stage disease, to monitor the progression of disease and to help plan treatment.

However, it has been demonstrated that there is regular error in the field of medical diagnostics. A review by the US National Academy of Medicine (2015) estimated that most people will experience at least one diagnostic error in their lifetime, sometimes with devastating consequences.

Available data, often subject to confidentiality, is insufficient to extrapolate a specific estimate of incidence or range of diagnostic error.

In the field of medical imaging, inappropriate or suboptimal choice of imaging test can be both harmful and/or delay diagnosis and/or result in misdiagnosis.

The US Food and Drug Administration (FDA) has estimated that 20%-50% of high-tech imaging procedures, such as CT scans, fail to provide information that improve individual person welfare and may represent, at least in part, unnecessary imaging services and cost.

The John Hopkins Armstrong Institute for Patient Safety and Quality reviewed 11,592 diagnostic error malpractice insurance claims filed between 2006-2015. They found that three disease categories (cancer, vascular events, and infections) accounted for 75% of all serious harm from diagnostic error (that is errors leading to death or serious, permanent disability): misdiagnosed cancers comprised 37.8% or diagnostic errors, vascular events 22.8% and infections 13.5%. They further observed that, apart from the detrimental effect of a confusion of diagnostic ‘codes’, about 85% of the misdiagnosed cases was attributable to failures of clinical judgment.

The affected population is diverse. Among diagnostic-error cases of varying severity, the median age of the individuals with errant diagnoses was 49, and more than half were female. For children and young adults (0-20 years old), harm was most often from missed infections (27.6%) rather than vascular events (7.1%) or cancers (9.1%), while the reverse was true for middle-aged and older adults.

About half of the high-severity harm cases the researchers studied resulted in patient death, while the other half resulted in permanent disability. Most of the diagnostic errors (71.2%) associated with the malpractice claims occurred in ambulatory settings for example, emergency departments (where missed infections and vascular events such as strokes were more of a concern), or outpatient clinics (where misdiagnoses were more likely to be cancer-related).

Proposed solutions comprise deploying computer-based diagnostic decision support tools, routine diagnostic performance feedback for clinicians and improving diagnostic education through simulation training.

Clinical decision support (CDS) systems help to inform a practitioner at the point of referral. Such clinical decision support (CDS) systems are based on professional guidelines: for example, the UK Royal College of Radiologist (RCR) guidelines, the European Society of Radiology (ESR) Guidelines and the American College of Radiology (ACR) Appropriateness Criteria (US AUC).

The UK Royal College of Radiologists (RCR) CDS (iRefer™) comprises a synthesis of evidence-based guidelines from the UK and international sources to make recommendations for everyday use of clinical imaging services.

The ESR guidelines (iGuide™) are largely based on the Appropriateness Criteria from the ACR whereby the ACR criteria are adapted for European users by the European Society of Radiology (ESR) for each new release.

The US AUC was initiated following the Protecting Access to Medicare Act (PAMA) of 2014 to increase the rate of appropriate advanced diagnostic imaging services provided to Medicare beneficiaries. Under the US AUC, at the time a referrer/practitioner orders an advanced diagnostic imaging service (for example, CT, PET, nuclear medicine, and/or MRI) for a Medicare beneficiary, the referrer/practitioner, or clinical staff acting under his/her direction, are required to consult a qualified Clinical Decision Support Mechanism (CDSM). The CDSM provides a determination of whether the order adheres to the US AUC, or if the US AUC consulted was not applicable (e.g., no US AUC is available to address the patient's clinical condition). Referrers/practitioners whose ordering patterns are considered outliers are subject to prior authorization.

US AUC thus impacts all advanced diagnostic imaging services whose claims are paid under the physician fee schedule, hospital outpatient prospective payment system or ambulatory surgical center payment system.

While targeted use of such systems has been associated with a decrease in the inappropriate utilization of advanced imaging tests, the current CDS systems are still limited in their usefulness. For example, CDS systems are often made available to referring clinicians in print or via standalone digital portals. The lack of integration between CDS systems and referring systems means that physicians/radiologists rarely consult with the appropriateness guidelines when creating referrals in such circumstances.

Furthermore, current appropriateness guidelines are determined by a mixture of in-depth review of scientific literature and consensus opinions from leading experts in radiology. This is a labour-intensive process which limits the number of radiology tests for which appropriateness criteria are available leaving swathes of radiological procedures without appropriateness guidelines, due to an absence of meaningful data.

Furthermore, the current CDS systems can be considered as ‘static’ models: that is, after they are published, they remained unaltered until their next scheduled update. The US ACR and the ESR guidelines are updated every 12-24 months; the RCR updates their guidelines every 5 years. The pace of these updates runs contrary to the dynamic pace of evolution in medical diagnostics.

Furthermore, the guidelines produced by the US ACR, ESR and RCR do not change in a data driven manner, based on the location or context of their use, despite the presence of wide variations of disease prevalence and incidence in different parts of the world due to different genetic, dietary, social, ethnic, education, environmental and healthcare factors etc. For example, in some European countries, the prevalence of cancer is higher in the younger age group (25 to 59 years) amongst those with lower education. In this example population, given the cancer risk is higher, certain imaging tests would be appropriate that would not be as appropriate in regions where the cancer risk was lower in the younger age group.

There are also stark differences in the prevalence of certain conditions depending on location. For example, in Denmark, the prevalence of patients presenting with migraine/headache is 16 times more common than in the UK, yet the same guidelines are provided for imaging of headaches regardless of this significant disparity.

This one-size-fits-all approach limits the effectiveness of the CDS systems, misses the opportunity to provide more bespoke data driven guidelines and thereby increase the accuracy and efficacy of diagnostic imaging.

Means are known whereby practitioners can access/receive information on propensity of medical conditions at a general population level based on medical and non-medical patient data; to identify risk of disease via a probability threshold based on retrospective image analysis; to use artificial intelligence to adjust weights, risk thresholds and variables in response to trends in individual person data; and to use image analysis to categorise disease status.

The present invention improves on these means.

### SUMMARY OF THE INVENTION

According to a first aspect of the invention there is a clinical decision support system comprising all features of claim 1. There may be a method for selecting a clinical pathway using a clinical decision support system comprising all features of claim 1. In this way the practitioner may be provided with a patient specific prospective usefulness of a referral: for example to guide a subsequent diagnostic imaging referral. Further features of the invention are disclosed in dependent claims.

The clinical decision support system may comprise a data input means and memory to receive and store a first group of data specific to at least one individual. The data input means and memory may be configured to receive and store a second group of data related to health conditions. The clinical decision support system may comprise a data analyzer to relate the first group to the second group to infer a specific propensity of the individual(s) to a health condition.

It is an advantage of the clinical decision support system that it can be procedure, indication or diagnosis driven. It is a further advantage that the prediction may be used to vet and protocol the referral against the CDS results/guidelines; and the outcome data may be used to inform and dynamically update the CDS reference criteria, including pathological input to diagnostic guidelines.

The Clinical Decision Support System may provide a practitioner with patient-specific indication of prospective usefulness of findings for a referral. Comparative and statistical input data may be supplemented by nuanced data to predict the usefulness of findings from optional and/or select subsequent diagnostic tests relative to patient specific propensity for a condition/disease. The data analyzer may be configured to generate a first list of medical tests relevant to the health condition.

The data analyzer may be configured to relate the first and second group to each one of the medical tests and provide a probability of a particular result respective of each one of the medical tests. The prediction thus informs the practitioner prior to the practitioner selecting e.g. the next diagnostic test, enabling the practitioner to modify and supplement their test selection based on the predictive usefulness outcome. The data analyzer may be configured determine a probability of usefulness of the particular result of each one of the medical tests. It may be configured to generate a hierarchy of usefulness of the tests based on a probability of usefulness of the particular result.

Among other advantages, the present clinical decision support system and method improves the efficacy of the selected test; improves the accuracy of associated diagnosis; avoids and/or reduces the incidence of unnecessary imaging procedures; and minimizes patient exposure to adverse events (including radiation exposure, contrast agents and misdiagnosis).

In an embodiment the system and method are integral to a clinical decision support platform.

It is an advantage of the present clinical decision support system and method that it can be procedure, indication or diagnosis driven.

Input patient data may include patient demographic, medical and non-medical data including geographic, near location and biomarker, including seasonality biomarkers.

The intelligent clinical decision support system ‘i’“CDS’ or intelligent CDS system provides means whereby referring practitioner(s) from inside and/or outside a network can directly access appropriateness guidelines; can vet and protocol the referral against CDS results/guidelines; and the comparative outcome data may be used in turn to dynamically update reference criteria and thereby the CDS guidelines.

The data analyzer in the clinical decision support system may be configured to parse structured data from unstructured medical reports in the second group of data. AI technology may be used to parse structured data from the unstructured medical reports, for example radiology reports. Machine learning may be applied and deep learning may be applied to analyse the outcomes, including comparative analysis with patient-specific information such as demographics, clinical indications, lifestyle, location, medical history, genetic factors, medication, seasonality, biomarkers and so forth.

The ‘CDS’ may predict the likelihood of a specific diagnosis being provided by a specific diagnostic test at the point of referral.

The outcome data may be integrated in a pathology step whereby a diagnostic approach may be decided for a patient given their demographics, clinical indications, lifestyle, location, medical history, genetic factors, medications etc. where the diagnostic may be imaging and/or pathology.

In an embodiment, the ‘CDS’ informs procedure whereby: 1/practitioner reviews nuanced patient data plus a selected test plus external non patient specific data (seasonal variation etc.); 2/clinical indications (e.g. headache post trauma) are input; 3/a probability of findings/diagnoses via the selected test are generated plus other relevant available tests; 4/a hierarchy of usefulness of the tests may be generated, compared to the chosen test, based on the probability of the findings/diagnosis of concern being identified by the test related to the patient data plus clinical indications plus external non patient specific data; 5/the practitioner chooses a test informed by the hierarchical results; 6/the selected test may be analysed and a report may be produced; 7/the findings of the diagnostic report are fed back into the ‘CDS’ to enhance and refine future assessments of patients in a similar circumstance. At least one of the tests may be selected and performed, usefulness of the result(s) may be noted and may be stored in the memory. The first list of medical tests and usefulness of the test result(s) may be fed back to the data analyzer.

In an embodiment, the ‘CDS’ may be indication driven whereby: 1/a probability of findings may be generated based on nuanced patient data plus clinical indications plus external non patient specific data (seasonal variation etc.); 2/a probability of findings of all relevant tests related to the input information may be generated; 3/a hierarchy of usefulness of the available tests may be generated based on the probability of significant diagnoses/findings being identified by the test relating to the patient data plus clinical indications plus external non patient specific data; 4/the practitioner selects a test, informed by the hierarchical results; 5/the report produced by the test selected may be analysed; 6/findings of the diagnostic report are fed back into the ‘CDS’ to enhance and refine future assessments of patients in a similar circumstance.

In an embodiment, the ‘CDS’ may be diagnosis driven whereby: 1/nuanced patient data plus suspected diagnosis (e.g. lung cancer) plus external non patient specific data (seasonal variation etc.) are input; 2/a probability of findings/diagnosis of all relevant tests may be generated related to the information input; 3/a hierarchy of usefulness of the tests may be generated based on the probability of the suspected diagnosis/findings being identified by the test; 4/practitioner selects the test informed by the hierarchical results; 5/a report, produced by the selected test, may be analysed; 6/findings of the diagnostic report are fed back into the ‘CDS’ to enhance and refine future assessments of patients in a similar circumstance.

In an embodiment the data may be available to a patient via a patient-facing CDS to improve patient understanding of diagnostic and pathology requirements.

In an embodiment, the ‘CDS’ collates imaging, diagnostics, pathology, cardiology and neurology and the scope, quality, and clinical pertinence of the data and as such integrating otherwise siloed knowledge.

In an embodiment the ‘CDS’ may be adjunctive to clinician's first referral (in lieu of radiologist) and/or informs radiologist approval of referral and/or helps interpretation of findings.

The proposed clinical decision support system may be a complex system which aggregates multi-modal data and deals with uncertainty, both in its input and output. Thus the system proposed needs to have flexibility to incorporate multi-modal data at any stage as more evidence may be being created, while at the same time being able to compare instances of data which do not contain the same level of information and were captured at different periods of time. While a subset of the data from patients do not vary much over time, there may be a set of dynamic data which varies during the life of a patient which may not be captured periodically when transitions (i.e. practitioner's referrals) in the clinical pathway occur.

Therefore, the decision support system may consider the current and past state of patients and the transitions between states while at the same time assuming that not all states and transitions are captured.

In a preferred embodiment the present invention comprises the steps of:
Step 1. A practitioner reviews nuanced patient data, and/or selected test data, and/or diagnosis data and/or clinical indication data, along with external non-patient specific data e.g. seasonal trends (patient data, test data, diagnosis data, clinical indication data and external data are referred to hereafter collectively as ‘data’);
Step 2. A comparative probability of findings/diagnosis of all relevant tests is generated related to the information input;
Step 3. A hierarchy of usefulness of the tests is generated, based on the probability of usefulness of findings/diagnosis;
Step 4. A report is generated, analysed and the practitioner selects the test informed by the hierarchical results
Step 5. The findings of the diagnostic report are fed back into the CDS to enhance and refine future assessments of patients in a similar circumstance.

One or more or all of steps 1 to 5 may be realised by structuring the data in the CDS as a graph. For example an overlay graph may be informed by steps 1, 2, 3 and 4, or 2, 3 and 4, or steps 2 and 3. Other combinations of the steps are possible to inform the graph. Some or all of the steps may be conducted together and/or consecutively. A step may be removed after one or more iterations.

In the clinical decision support system the data analyzer may be configured to generate a State-Transition chain in which states of the individual at respective times are interleaved by a transition made by a practitioner. The transition may be temporality intermediate one of the states and the next. The transition may be conceptually intermediate or feature-wise intermediate one of the states and the next. The transition may be directly intermediate the states in the State-Transition chain or there may be other states temporally, conceptually, or feature wise in between.

The clinical decision support system may comprise a display in communication with the data analyzer and memory to provide a visible graph of the State-Transition chain(s).

State (Node), refers to state of the patient at a particular given time. It may be defined by multiple multi-modal features containing all information available about the individual at that particular time (both clinical and non-clinical data). It may be modelled as a node in the graph.

The data input means and memory may be configured to receive and store the states in the first group of data. Each state comprises multiple multi-modal containing information about a respective one of the individuals at the respective time.

Transition (Edge), refers to the referral/actions given by the practitioner to progress from one State into another. It may be defined by two well defined features, i.e. the diagnosis and the set of actions recommended by the practitioner. It may be modeled as an edge in the graph.

The data input means and memory may be configured to receive and store each transition in first group of data. Each transition may comprise a diagnosis by the practitioner based on a state before the transition and an action or actions recommended by the practitioner based on the diagnosis.

The graph may be composed as a collection of State-Transition chains of undetermined length which may be given by the past information available for each patient. As such there may be multiple unconnected graphs related to the same patient in the overall structure of the graph.

The data analyzer may be configured to form in the memory at least one cluster of the states, wherein each of the states in the cluster comprises data specific to a different individual and the data for each state may be similar. The data analyzer may be configured to include states in each cluster which comprise data specific to a different individual. The data analyzer may be configured to include states in each cluster which comprise data specific to a different practitioner.

Step 1 may be effected by means of presenting the practitioner with the new set of multi-modal features representing the current State of the patient, as well as the list of historical States and Transitions. This information represents the most up-to-date information for that patient and is presented as a collection of structured and unstructured data tagged by time and embedded in a graph such that the practitioner can easily navigate the temporal structure in the form of States and Transitions and their associated multi-modal data which contribute to form the input data.

The different types of multi-modal data may be predefined and linked to the structure of the State and Transition. The core features may be mandatory. The patient's

States-Transitions representation will be populated with information from databases and other sources via a parser which will extract the key information.

The practitioner may be given the option to select which data is to be inputted in the system as well as prompting the requirement for mandatory data which needs to be populated for the system to provide a comparative probability. This system may be a graphical interface to review and select which data is to be inputted to the system initially for querying and subsequently for reinforcement learning purposes.

Step 2 may be effected by means of calculating similar States, i.e. Nodes with similar values in their features representing a particular state of a patient, may be shown in a group or the cluster in a virtual overlay close to each other. The graph may be queried for similar States when looking to predict the next point of referral (Transition). This virtual overlay may be generated using feature embeddings which may enable to calculate the distance between nodes. Different embedding techniques can be used however the use of Transformer architectures with aggregated embeddings permit the fusion of multi-modal data. This network will be pre-trained with historical data to generate embeddings structures.

The data analyzer may be configured to quantify similarity of each state relative to the other states, and to place states with a similarity above a particular level in a cluster. The data analyzer may be configured to assign a particular weight to a particular one of the data specific to at least one individual to influence the similarity of each state relative the other states.

Different weights may be assigned to each feature to include attention mechanisms according to the practitoners' taxonomy of each feature to emphasize the importance of key input data features. So that there is a potential to have or create multiple virtual overlays for enhanced decision making.

The data input means and memory may be configured to receive and store the weight as assigned by a practitioner. The data input means and memory may be configured to receive and store the weight as assigned by the data analyzer parse of data in the first group and/or the second group. Querying the graph, once Step 1 is realised with data from a new patient, may be required to discover the closest States to that patient in the virtual overlay connected by the required Transitions. This may be achieved by using graph embeddings containing both the State and Transition embeddings as a vector space to which a similarity metric can be derived from; note that a range of distance metrics for vector spaces are available in the literature, e.g. cosine similarity.

Navigating the overlay of embeddings, rather than the graph itself, may be a computationally efficient method which may escalate well as more nodes/edges are incorporated in the graph.

The data analyzer may be configured to overlay the cluster(s) to clarify which states are within which cluster. Each state may be labeled with a visible tag by date or time of data associated with the state. The data input means may comprise a touch sensor on the display to detect a touch of the display where a state may be displayed. The data input means may comprise another means to manipulate a screen cursor to a position where the state may be displayed to glean information from the display about the state. The data analyzer may be configured to display data associated with the state where the display may be touched or the cursor location may be selected with a mouse for example.

The prediction of the referral may be then provided by finding the set of most similar subgraphs (pathways)—limited by a threshold considering a minimum and maximum similarity score per States and linked Transitions to prune the available options to explore as potential pathways—and identifying the set of next available Transitions in the graphs. For this purpose, a measure of certainty in the prediction of the next referral, i.e. next Transition in the graph, may be given as the similarity measurement of the graph containing the patient's current State and previous States-Transitions against the subset of identified potential matching graphs withing the overall graph. The current State as well as the immediate previous Transition may be given a higher weight (exponential weight) in the overall certainty score.

A limitation on the number of previous States and Transitions for querying and similarity analysis may be controlled with a threshold variable which is set empirically depending on the average number of States in the network per patient and the certainty score, and which may be configured based on the outcome of the matching graph process when querying.

Step 4 may be effected by predicting multiple transitions with their associated values of certainty which may be presented to the practitioner/clinician for decision making, together with an explanation of the key features impacting the prediction in the current State and previous States-Transitions for transparent decision making. This may be achieved via a graphical interface which will also accept the decision of the practitioner as a set of diagnosis and actions to be conducted on the patient, i.e. future Transition.

Step 4 may be effected by means of using the feedback function via the graphical interface. The graph may be reinforced with the feedback from the practitioner, i.e. with the selected next Transition, but only incorporating the next Transition once it has been “validated”: a transition may be marked as “provisional” once the practitioner provides his decision and may be marked as “validated”, once a State node may be created with information about the outcome of the Transition. This step may depend on the actions of the referral to be executed and validated by a practitioner which may give rise to a new State to link with the Transition. For querying purposes, the “Provisional” transitions may not be part of the graph.

According to another aspect of the invention there is a method of selecting a clinical pathway using the clinical decision support system disclosed herein.

The invention will now be described, by way of example only, with reference to the accompanying figures in which:

### BRIEF DESCRIPTION OF THE FIGURES

FIG. 1 shows a generic overview of an intelligent Clinical Decision Support (CDS) System;

FIG. 2 shows a State-Transition Chain in a query subgraph;

FIG. 3 shows an Overlay graph; and

FIG. 4 shows a flow from Embeddings to Overlay graph.

### DETAILED DESCRIPTION OF THE INVENTION

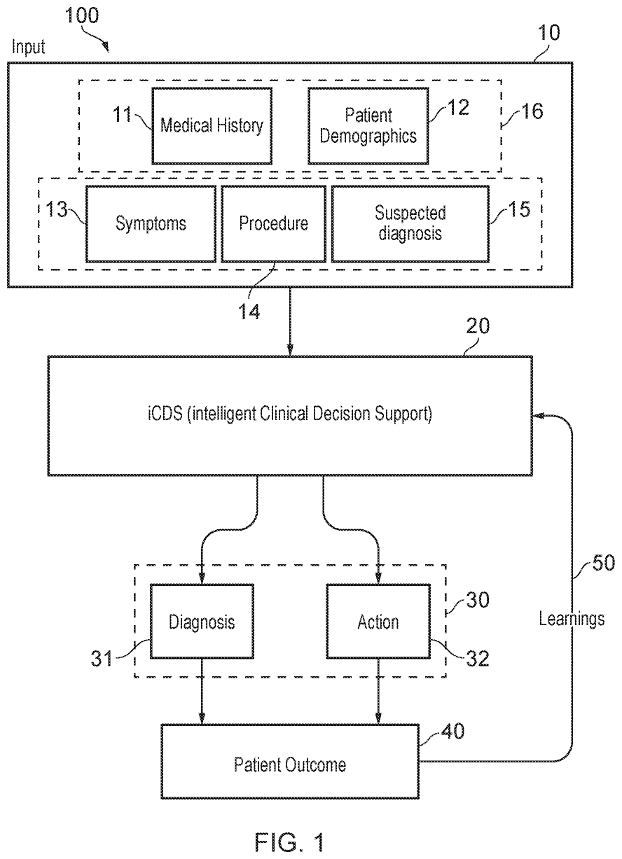

A clinical decision support system 100 is shown in FIG. 1. The clinical decision support system comprises a data input means and memory 10 to receive and store a first group of data 16. The first group of data is specific to at least one individual. Data in the first group includes medical history 11 of the individual(s) and demographics 12. Data in the second group of data is related to health conditions. Data in the second group includes information on symptoms 13, information on procedures to test for health conditions related to the systems 14, and information on suspected diagnoses 15.

The data 11, 12, 13, 14, 15 is passed from the memory to a data analyzer 20 which is configured to provide intelligent clinical decision support relevant to the data. The data analyzer 20 is configured to generate a State-Transition chain in which states of the individual(s) at respective times are interleaved by a transition 30 made by a practitioner. The transition 30 has two features or components. These components are a diagnosis 31 by a practitioner and an action 32. This diagnosis may be based on a consultation of the individual by the practitioner.

A patient outcome 40 follows the transition. Learnings 50 from the outcome 40 are fed back to the memory 10 and data analyzer 20 of the clinical decision support system.

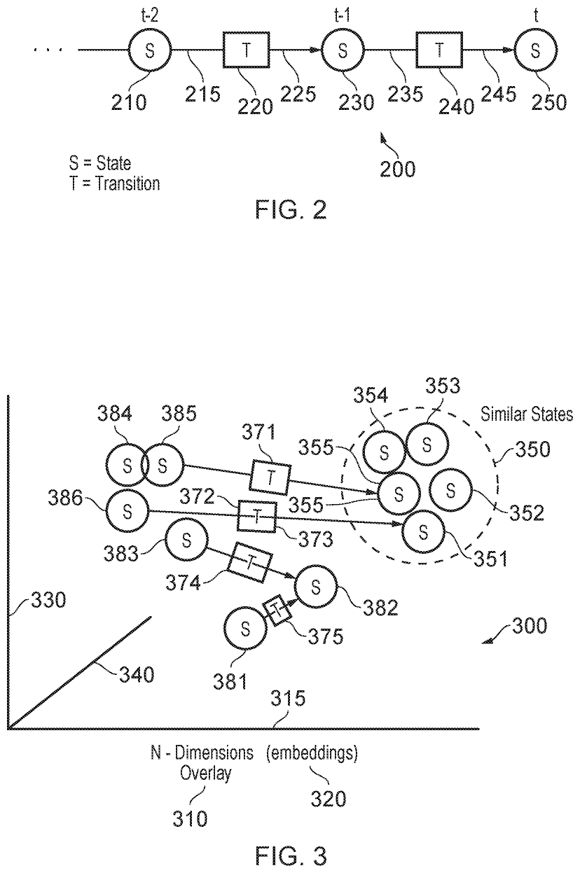

A portion of a State-Transition chain 200 is shown in FIG. 2. The portion of the State-Transition chain 200 comprises three states S 210, 230, 250. There is an early state S 210 at early time ‘t’ minus 2. There is a next to last state S 230 at next time ‘t’ minus 1. There is a current state S 250 at time ‘t’.

The three states S 210, 230, 250 are chained together by two transitions T 220, 240. The first transition T 220 is intermediate the early state S 210 and the next to last state 230. A first link 215 connects the early state 210 to the first transition T 220. A second link 225 connects the first transition T220 to the next to last state 230. The second transition T 240 is intermediate the next to last state S 230 and the current state 250. A third link 235 connects the second transition T 240 to the next to last state S 230, and a fourth link 245 connects the second transition T 240 to the current state S 250.

FIG. 3 shows an Overlay graph 300. There are N dimensions 310 (embeddings 320). Overlay graph has a horizontal axis 315, a vertical axis 330, and a diagonal bisector 340 from the axes' origin intermediate the vertical axis 330 and the horizontal axis 315.

Eleven states 351, 352, 353, 354, 355, 381, 382, 383, 384, 385, 386 are on the Overlay graph 300. There are three State-Transition chains on the Overlay graph 300. The first State-Transition chain on the Overlay graph 300 comprises state 381 chained by transition 375 to state 382, and state 382 chained by transition 374 to state 383. The second State-Transition chain comprises state 386 chained by transition 372 to state 351. The third State-Transition chain comprises state 385 chained by transition 371 to state 355.

There is a cluster 350 of five of the states 351, 352, 353, 354, 355 on the Overlay graph 300. The cluster 350 is surrounded by a visible dashed line to help a person using the Overlay graph 300 to spot the cluster 350. The net value of the embeddings of each of the five states 351, 352, 353, 354, 355 associated with the horizontal axis 315 of the Overlay graph 300 are similar within the cluster. Also the net value of the embeddings associated with the vertical axis 330 of the Overlay of each of the five states 351, 352, 353, 354, 355 within the cluster 350 are similar. So the five states 351, 352, 353, 354, 355 in the cluster 350 appear close together on the Overlay graph 300. Two of the state 351, 355 with the cluster 350 are chained by transitions 371, 372 to states 386, 386 outside of the cluster 350.

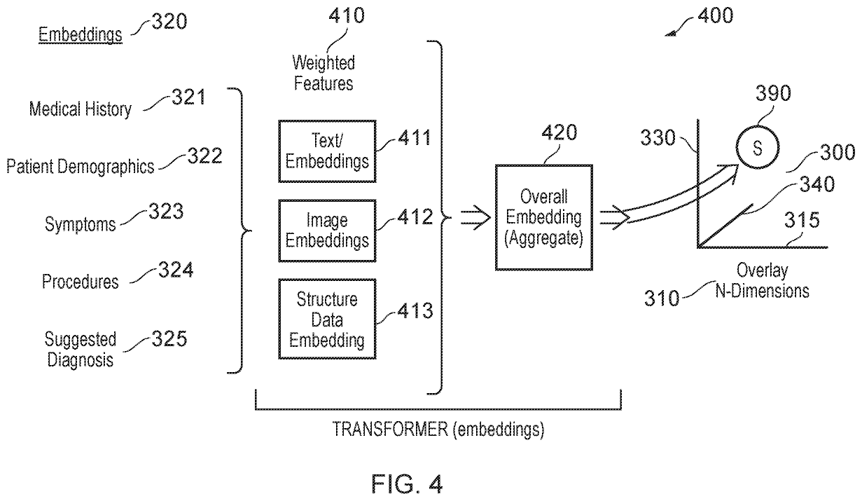

The net value of each state associated with horizontal axis 315 and the net value of each state associated with vertical axis 330 is affected by an amount of weight put on each feature. This is shown by FIG. 4 which illustrates a process of placing states 390 on the overlay graph 390. On the left side of FIG. 4 there is a column ‘embeddings’ 320. The embeddings include medical history 321, patient demographics 322, symptoms 323, procedures 324, and suggested diagnosis. Not one of these embeddings are essential. There could be other embeddings as this is only an example of many possibilities.

The second column from the left in FIG. 4 is weighted features 410. The weighted features 410 include text embeddings 411, image embeddings 412, and structure data embeddings 413. Not one of these embeddings 411, 412, 413 are essential, and others are possible. Each of the embeddings 320 in the right-hand column flow into the weighted features 410 in the second column. The embeddings 320 are weighted according to the weighted features 410, and then an overall embedding aggregate 420 is determined for each of the states 390.

In FIG. 4 states S 390 is indicative of a plurality of states to be shown on the Overlay graph 300. The net values of the states associated with the horizontal axis 315 and the vertical axis 330 are affected by the overall embedding aggregate 420. The location of the states S 390 on the Overlay graph 300 are therefore also affected by the overall embedding aggregate 420 and applied weight of weighted features 410. The weighted features 410 and overall embedded aggregate 420 taken together are the transformer embeddings 430.

By way of illustrative example, a 50-year-old male chronic smoker presents who is coughing up blood. The Intelligent Clinical Decision Support System ‘iCDS’ predicts the likelihood that a tumour, or pneumonia will be found on a chest radiograph (for example, 3% chance that a tumour will be detected, 10% chance that pneumonia will be detected) versus a CT chest (6% chance that a tumour will be detected and 25% chance that pneumonia will be detected). This probability data is presented to the practitioner at the point of care allowing them to inform their choice of test based on the likelihood of detection of the disease process that is most relevant to evaluate for. Thus, the practitioner avoids only referring for chest radiograph which will miss 50% of lung tumours, rather than CT chest which will identify the tumour if present, thus avoiding delayed diagnosis.

By way of further illustrative example, a 55-year-old female attends a consultation with her GP with new onset headaches over the past six weeks. She has a history of breast cancer and is currently on treatment. The GP wants advice on what diagnosis the available tests might identify in this scenario. ‘iCDS’ extracts patient and non-patient data from multiple sources including patient demographic, past medical history, diagnostic results, nutritional, environmental, geographic, and seasonal data etc. The ‘iCDS’ identifies 10% probability of patient having parenchymal metastatic disease and 10% chance of leptomeningeal metastatic disease. ‘iCDS’ identifies CT brain as having 2.5% probability of identifying parenchymal metastatic disease and 0.5% chance of leptomeningeal metastatic disease. The ‘iCDS’ identifies MRI brain with contrast as having a probability of 10% of finding parenchymal metastatic disease and 10% of finding leptomeningeal metastatic disease. The GP reviews ‘iCDS’ output and chooses MRI brain with contrast based on the increased probability of diagnosis of leptomeningeal metastatic disease compared to CT brain (20× more likely with MRI than CT). The scan is authorised because of the high likelihood of diagnosis. The MRI brain demonstrates leptomeningeal metastatic disease and the patient's treatment plan is updated. The data from this interaction is fed back into the model to keep refining the accuracy.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

1-24. (canceled)

25. A clinical decision support system comprising a data input means and memory to receive and store a first group of data specific to at least one individual and a second group of data related to health conditions, and a data analyzer to relate the first group to the second group to infer a specific propensity of the individual(s) to a health condition.

26. A clinical decision support system according to claim 1 wherein the data analyzer is configured to parse structured data from unstructured medical reports in the second group of data.

27. A clinical decision support system according to claim 25 wherein at least one of the tests is selected and performed, and the data analyzer is configured to:
generate a first list of medical tests relevant to the health condition;
relate the first and second group to each one of the medical tests and provide a probability of a particular result respective of each one of the medical tests;
determine a probability of usefulness of the particular result of each one of the medical tests; and
generate a hierarchy of usefulness of the tests based on a probability of usefulness of the particular result;
wherein the clinical decision support system is configured to note usefulness of the result(s) is/are noted, and feedback the first list of medical tests and usefulness of the test result(s) is/are fed back to the data analyzer.

28. A clinical decision support system according to claim 25 wherein the data analyzer is configured to generate a State-Transition chain in which states of the individual at respective times are interleaved by a transition made by a practitioner.

29. A clinical decision support system according to 28 wherein the data input means and memory are configured to receive and store the states in the first group of data, wherein each state comprises multiple multi-modal containing information about a respective one of the individuals at the respective time.

30. A clinical decision support system according to claim 29 wherein the data input means and memory are configured to receive and store each transition in first group of data, wherein each transition comprises a diagnosis by the practitioner based on a state before the transition and an actions recommended by the practitioner based on the diagnosis.

31. A clinical decision support system according to claim 29 wherein the data analyzer is configured to form in the memory at least one cluster of the states, wherein each of the states in the cluster comprises data specific to a different individual and the data for each state is similar.

32. A clinical decision support system according to claim 31 wherein the data analyzer is configured to include states in each cluster which comprise data specific to a different individual.

33. A clinical decision support system according to claim 31 wherein the data analyzer is configured to include states in each cluster which comprise data specific to a different practitioner.

34. A clinical decision support system according to claim 31 wherein the data analyzer is configured to quantify similarity of each state relative to the other states, and to place states with a similarity above a particular level in a cluster.

35. A clinical decision support system according to claim 34 wherein the data analyzer is configured to assign a particular weight to a particular one of the data specific to at least one individual to influence the similarity of each state relative the other states.

36. A clinical decision support system according to claim 35 wherein the data input means and memory are configured to receive and store the weight as assigned by a practitioner.

37. A clinical decision support system according to claim 35 wherein the data input means and memory are configured to receive and store the weight as assigned by the data analyzer parse of data in the first group and/or the second group.

38. A clinical decision support system according to claim 31 wherein different types of multi-modal data are linked to the structure of the state and transition.

39. A clinical decision support system according to claim 31 comprising a display in communication with the data analyzer and memory to provide a visible graph of the State-Transition chain(s).

40. A clinical decision support system according to claim 39 wherein the data analyzer is configured to overlay the cluster(s) to clarify which states are within which cluster.

41. A clinical decision support system according to claim 39 wherein the/each state is labeled with a visible tag by date or time of data associated with the state.

42. A clinical decision support system according to claim 39 wherein the data input means comprises a touch sensor on the display to detect a touch of the display where a state is displayed.

43. A clinical decision support system according to claim 42 wherein the data analyzer is configured to display data associated with the state where the display is touched.

44. A method of selecting a clinical pathway using the clinical decision support system according to claim 25.
